(19) United States
(12) Patent Application Publication
(10) Pub. No.: US 2005/0154323 A1
(43) Pub. Date: Jul. 14, 2005

(54) Response and locating system and a position indication marker device

(75) Inventors: Gereon Vogtmeier, Aachen (DE); Harald Reiter, Aachen (DE); Josef Lauter, Geilenkirchen (DE); Olaf Such, Aachen (DE); Christian Reichinger, Neutraubling (DE); Ralf Schmidt, Aachen (DE)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics N.V., BA Eindhoven (NL)

(21) Appl. No.: 10/509,234

(22) PCT Filed: Mar. 7, 2003
(86) PCT No.: PCT/IB03/00854

(30) Foreign Application Priority Data
Mar. 29, 2002 (EP) 02076228.2

(51) Int. Cl. A61B 5/04

(52) U.S. Cl. 600/509

(57) ABSTRACT

The invention relates to a response system for detecting, locating and responding to a person in an abnormal physiological condition, said system comprising positioning means (12) arranged to provide an information about an actual location of the person and monitoring means (11) arranged to monitor an abnormality in the physiological condition of the person, said monitoring means comprising a sensor (6) to acquire a signal related to a physiological condition of the patient, a processor (8) to process the acquired signal in order to establish where the abnormal condition has occurred, said processor being arranged to produce a trigger signal to generate an alarm, an alarm generating means (10) arranged to generate the alarm upon a receipt of the trigger signal, said alarm generating means being further arranged to provide an indication of a location of the person said indication of the location being provided by a positioning means (12). In order to provide a valid position information of the user in case of an emergency the system comprises further a position indication marker (PIM) device (14) arranged to store the valid position information in a storage unit (13). The PIM device is arranged to download the position information as a co-ordinate, address or any other suitable description of the position of the user and to transmit said information to the monitoring means in case of the emergency.

The invention relates to a response system for detecting, locating and responding to a person in an abnormal physiological condition, said system comprising positioning means arranged to provide an information about an actual location of the person and monitoring means arranged to monitor an abnormality in the physiological condition of the person, said monitoring means comprising a sensor to acquire a signal related to a physiological condition of the patient, a processor to process the acquired signal in order to establish where the abnormal condition has occurred, said processor being arranged to produce a trigger signal to generate an alarm, an alarm generating means arranged to generate the alarm upon a receipt of the trigger signal, said alarm generating means being further arranged to provide an indication of a location of the person said indication of the location being provided by a positioning means.

The invention further relates to a portable position indication marker device.

A response and locating system is known from U.S. Pat. No. 6,292,687. The known system is arranged to activate a personal alarm upon an event of a medical emergency, for example a cardiac arrest. The known system comprises monitoring means for detecting the medical emergency together with positioning means arranged to provide an information upon an actual location of the victim to remotely located medical personnel. The latter is achieved in the known system by means of localization signals. In the known system the localization signals include signals from a global positioning system or other signals deduced from an established local network of position localizers. Such position localizers may be implemented as a set of transmitters with known co-ordinates or location, arranged in public places, like hotel rooms or airport halls.

The known system has a disadvantage in that it provides the position information only in cases a connection to the global positioning system is not interrupted or in cases when an interior of buildings is equipped with a network of position localizers.

It is an object of the invention to provide a response system, which provides an accurate position information even in cases no connection to remotely set-up localizers is possible.

The response system according to the invention is characterized in that the system further comprises a portable position indication marker (PIM) device, the positioning means being arranged to communicate to the PIM device, said PIM device comprising a storage unit to store a position information and transmitting means to transmit the position information to the positioning means. According to the technical measure of the invention the response system is equipped with a position indication marker (PIM) device is arranged to store the position information in form of an actual address, or a latitude-, longitude-, height-information or in another form of unambiguous description. The PIM device is portably arranged and can be worn by a patient together with the response system. For example, in case a patient is suffering from a heart failure, the response system comprises heart monitoring means arranged to monitor a heart function, which are arranged to transmit an alarm together with the position information. The position information is acquired by the positioning means from the PIM device, where it is stored or pre-stored. Thus, the response system can always provide an up-to date information upon the location of the patient, even for locations, where a global positioning system (GPS) on any other satellite navigation system is not reachable and where no infrastructure of terrestrial localizers is installed. Next to the above, the system according to the invention has the advantage that by using the PIM device a reduced power consumption by the system is achieved by switching off a satellite reception in case the pre-stored PIM information is sufficient. Further, the localization by means of PIM can be more accurate than the information provided by means of a satellite navigation system, especially if a full address is used as the position information. For example, if a person stays at home, office or at friends with a known address there is no need to use the satellite navigation system to calculate his dwell position.

An embodiment of the system according to the invention is characterized in that the position indication marker device is arranged to download the position information from a satellite navigation system by means of an interface. In case the response system has detected the abnormality in the physiological condition of the person, it will search for a position identification. In case the person is outside his home or another known dwelling, the positioning means address the PIM device and the PIM device forwards a co-ordinate request to the satellite navigation system, for example a GPS or any other satellite navigation system. Upon the receipt of the co-ordinates from, for example, the GPS, the PIM device transmits the co-ordinates to the positioning means. Both connections can be carried out by means of a wire-less connection. It is also possible that the PIM device is integrated in a mobile station comprising both a satellite interface and a GSM electronics. In this case the user has a limited number of devices on him, which ensures better privacy and user-friendliness of the system as a whole. The PIM device can be further arranged to automatically store the last position known from the satellite navigation system before the connection to said system is interrupted and to use this last position further as a last valid position information for the positioning means.

An embodiment of the system according to the invention is characterized in that the position indication marker device is further arranged to download the position information from a terrestrial positioning system. In cases no satellite signal can be found, the patient can still be in an area with a pre-installed network of terrestrial position localizers, such as a system of RF transmitters or beacons in airport halls or other appropriately arranged public places. In this case the PIM device contacts the transmitters of the local network, picks up an appropriate signal identifying the position and further makes it available as an appropriate signal to the positioning means. For this purpose the PIM device is arranged to comply with broadcasting formats conventionally used by such local network of transmitters. The advantage of this technical measure in that the user does not have to manually program the position information in the PIM device while entering the areas which have an installed network of terrestrial position localizers.

An embodiment of the system according to the invention is characterized in that the position indication marker device is further arranged to download the position information from a pre-set table of dwell positions stored in the storage unit. The storage unit can be a conventional programmable memory block, where the user can put an alpha-numerical information describing his dwelling, for example by means of a keyboard. In case the user is frequenting a certain dwell positions, like office, theatre, friends, etc, the actual dwell position can be chosen form a pre-set table stored in the storage unit. This offers a convenient tool for the user to register his position. It is also possible that the PIM device is arranged to make a PC connection, so that a list of dwell positions can be downloaded instantly from a database. Also, the PIM device can be arranged to communicate to a Personal Digital Agenda (PDA) or any like system and to store and update the position information of the user in the storage unit based on the PDA inputs. In case of an emergency, the PDA-inputs can be used to provide position information, which is especially advantageous if no further information about the position of the user is available. This improves the user-friendliness of the system even further.

The portable position indication marker (PIM) device according to the invention is characterized in that said device comprises a position deriving means arranged to derive the position information, a storage unit to store the position information, an identification unit arranged to respond to a linked-up positioning system and a transmitter to transmit the position information to the linked-up positioning system. Such a PIM device can be used as a stand-alone system in areas where no connection to a satellite navigation system can be established. If a positioning system of any kind is searching for a location co-ordinate, it can link-up to the PIM device and download the pre-set co-ordinate or address information. Such a PIM can be preferably used in large conference halls or at mega-cinemas, for example. The stand-alone PIM can be arranged with a keyboard and display in order for a user to be able to introduce new information or to edit the present information. Alternatively, the PIM device can be arranged with a chip-reader, where a position programming takes place by means of programming an insertable chip into the PIM device. Optionally, such a PIM device can be equipped with a display to control the input information.

These and other aspects of the invention will be discussed with reference to the attached figures.

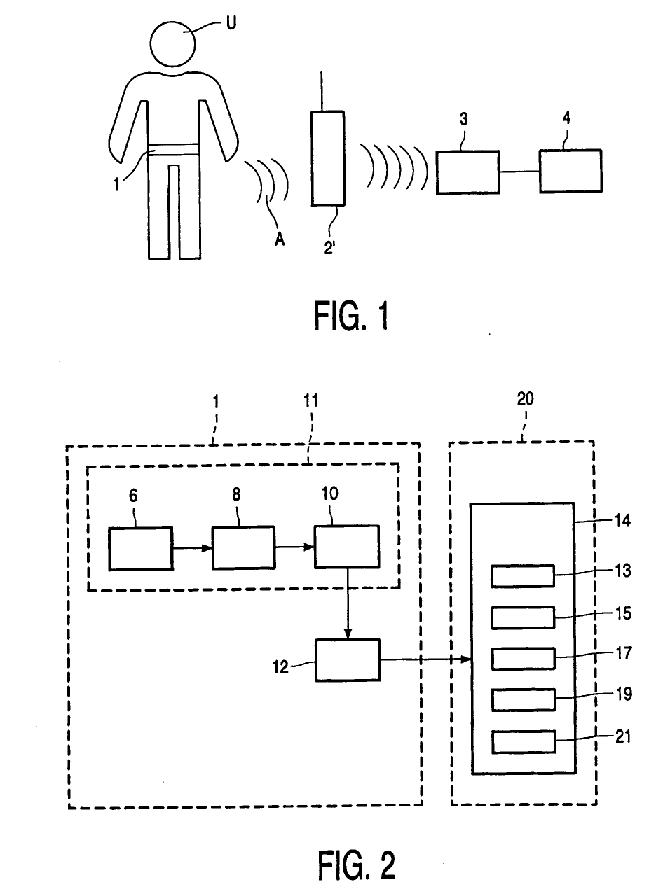

FIG. 1 shows a schematic view of an embodiment of the response system architecture in case the user is located outside.

FIG. 2 shows schematically an embodiment of the components of the user-side of the system according to the invention.

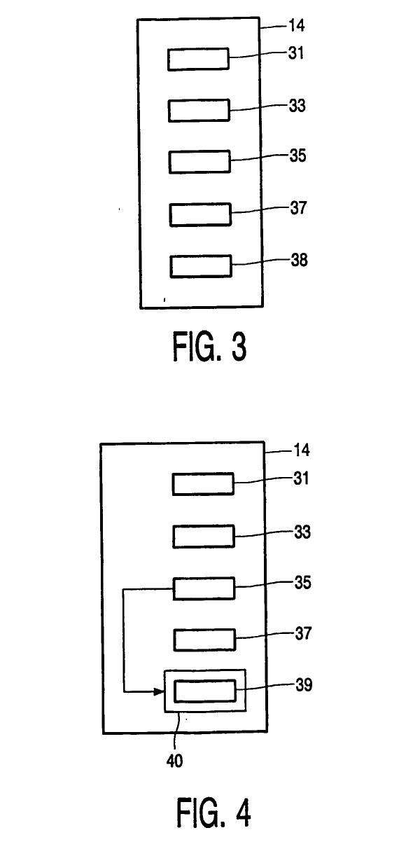

FIG. 3 shows schematically an embodiment of the position indication marker (PI) device according to the invention.

FIG. 4 shows schematically an embodiment of the PIM device 14 provided with a user interface to input a pre-known position information.

FIG. 1 shows a schematic view of an embodiment of the response system architecture according to the invention in case the user is located outdoors. The user U is provided with a detection and alarm system 10 comprising a user-side 1 and a non-user side 2′. The user-side I comprises monitoring means and positioning means [both not shown]. The user-side 1 of the system 10 is arranged to transmit an alarm signal A together with position information, preferably via a RF-link to the non-user side 2′ of the system. The non-user side 2′ comprises a mobile station 2′ arranged to pick-up the alarm signal A and the position information and to forward them to a remote service center 3 by means of a wireless connection. An example of a suitable transmitting technology is GSM for the mobile station. The remote service center 3 is arranged to handle the medical emergency, for example by forwarding the alarm signal to a public emergency center 4.

FIG. 2 shows schematically an embodiment of the components of the response system according to the invention. The user-side 1 comprises monitoring means 11 arranged to monitor an abnormality in the physiological condition of the user. The monitoring means 11 comprise a set of electrodes 6 arranged on the body of the user to pick-up a signal characteristic to the physiological signal, for example an ECG signal, a body temperature, respiration rate, encephalogram, etc. Additionally, the monitoring means 11 can comprise another sensor [not shown] arranged to monitor a signal not directly related with a targeted physiological condition. An example of such a sensor is a motion sensor, or a blood pressure sensor. The user side 1 further comprises positioning means 12 arranged to provide an information about the actual location of the person being monitored. The electrodes 6 are arranged to provide the signal to the processor 8 of the monitoring means 11 of the system 1. The processor 8 is arranged to generate a trigger signal in case an abnormality in the condition being monitored is detected. The trigger signal is then forwarded by the processor 8 to the alarm generation means 10. The alarm generating means 10 address the positioning means 12 in order to acquire the actual position of the person. The positioning means 12 are arranged to communicate to a portable position indication marker (PM) device 14 in order to acquire the actual position of the person. In its most simple embodiment the PIM device 14 comprises a storage unit 13 arranged to store the position information, and a transmitter unit 15 to transmit the position information to the positioning means 12, preferably on request, and a micro-processor 19. Such an implementation of the PIM device suffices in situations when the dwell position of the person is known and is not being changed for some time, for instance when a person is at his friends, at cinema, at his office or at other locations with a reasonable dwell time. The information about the actual dwell position is thus pre-known and can be pre-stored in the storage unit 13 of the PIM device 14. When the person arrives at one of such pre-stored locations, its co-ordinate or an address can be selected in the PIM and can be made available as an appropriate signal to the positioning means 12.

Additionally, in case the person is changing his dwell position, the PIM device 14 can comprise an interface 17 to a satellite navigation system, for example a global positioning system (GPS) or any other system providing position information, for example GSM, UMTS, etc. in order to update the position of the person. In case the abnormality in the physiological condition does occur while the person is changing his dwell position, the positioning means 12 link-up to the PIM device which supplies the actual position information obtained via the GPS interface 17.

To cover for situations, when the person is changing his dwell position from a mobile outside position with a working GPS reception to an in-door position with no GPS reception, the micro-processor 19 stores the last actual GPS co-ordinate before the transmission was interrupted as an actual position information in the storage unit 13. This can be technically implemented, for example by providing a ‘store’-button on the PIM device, so that the user can store the last known GPS position in the storage unit as the actual position information. In case a building is provided with a network of terrestrial localizers, the PIM device 14 initiates a beacon receipt unit 21 to communicate with the beacons sent-out by the network of terrestrial localizers. It is also possible that the unit 21 is arranged to communicate to terrestrial RF-signals, like GSM networks. Alternatively, both functions can also be integrated in the interface 17. In both cases the dwell position of the person inside the building will be updated and will be used as the position information for the positioning means in case of an emergency.

For the convenience of the user the PIM device 14 can be integrated in a mobile telephone 20. In this case the number of separate devices to be kept by the user is minimized.

FIG. 3 shows schematically an embodiment of the position indication marker (PIM) device according to the invention. The PIM device 14 comprises a storage unit 31 arranged, for example as a programmable memory to store the position information, an identification unit 33 arranged to identify and to respond to a positioning system attempting to link to the PIM device, position deriving means 35 arranged to derive the actual position of the user and a transmitter 37 arranged to transmit the derived position information to the linked-up positioning system. The PIM device further comprises a micro-processor 38 arranged to control the operation of the PIM device as a whole. In case the identification unit 33 recognizes a positioning system attempting to link-up to the PIM device 14, the micro-processor 38 sends an appropriate signal to the position deriving means with a request of a valid actual position of the user. There are several operational scenarios possible. First, in case the user is located within a dwelling with a known address or co-ordinate stored in the storage unit 31, the position information is retrieved from the storage unit 31 by the position deriving means 35 and is transmitted by the transmitter unit 37 in a form of an appropriate signal to the linked-up positioning system. Secondly, in case the user is mobile with a not known dwell position, the position deriving means 35 attempt to link to an outside positioning system, for example by means of a satellite interface [not shown] or an interface to a network of installed terrestrial localizers [not shown] in case no satellite signal can be received. The actual position information retrieved from one of the above interfaces in then transmitted by the transmitter unit 37 in a form of an appropriate signal to the linked-up positioning system. Finally, in case the user is outside a known dwelling and no contact can be made to an external positioning system, the PIM device can address the position information which is made available to the PIM by the user himself by means of the user interface. This function is explained in more detail with reference to FIG. 4.

FIG. 4 shows schematically an embodiment of the PIM device 14 provided with a user interface to input a pre-known position information. The PIM device 14 comprises next to the units discussed with reference to FIG. 3 a user interface 39 comprising input means 40 to input alpha-numerical information. For example, the user can manually input addresses or co-ordinates in a table describing possible dwellings. An example of the input means 40 is an interface to a personal computer comprising a database of position information. Alternatively, the input means can be arranged to be operated by a voice control or via an SMS interface. After programming is accomplished, one dwell position can be chosen by the user via the user interface, said position being later made available to the positioning system by the transmitter unit in case of an emergency.

Thus, the PIM device being a portable apparatus constitutes reliable means to provide an unambiguous information about the position of the user on request. The operation of the PIM device according to the invention is independent of the access to external localizers resulting in a possibility to provide an accurate information about the location of the user anytime anywhere. Due to the programmable memory of the PIM device it is possible to implement the device with different possible levels of functionality ranging from a simple chip-card operated device for identifying a single location, to an elaborate device comprising interfaces to databases to download a plurality of possible dwell positions of the user and/or interfaces to external systems of localizers. Due to the PIM device the person suffering from a life-threatening condition can be found faster resulting in an increased chance for his survival.

1. A response system for detecting, locating and responding to a person in an abnormal physiological condition, said system comprising positioning means arranged to provide an information about an actual location of the person and monitoring means arranged to monitor an abnormality in the physiological condition of the person, said monitoring means comprising:
a sensor to acquire a signal related to a physiological condition of the patient;
a processor to process the acquired signal in order to establish where the abnormal condition has occurred, said processor being arranged to produce a trigger signal to generate an alarm;
an alarm generating means arranged to generate the alarm upon a receipt of the trigger signal, said alarm generating means being further arranged to provide an indication of a location of the person said indication of the location being provided by the positioning means,
characterized in that the system further comprises a portable position indication marker (PIM) device, the positioning means being arranged to communicate to the PIM device, said PIM device comprising a storage unit to store a position information and transmitting means to transmit the position information to the positioning means.

2. A system according to claim 1, characterized in that the position indication marker device comprises an interface arranged to download the position information from a satellite navigation system.

3. A system according to claim 1, characterized in that the position indication marker device is further arranged to download the position information from a terrestrial positioning signal.

4. A system according to claim 1, characterized in that the position indication marker device is further arranged to download the position information from a pre-set table of dwell positions stored in the storage unit.

5. A system according to claim 2, characterized in that the position indication marker device comprises a micro-processor arranged to validate said interface, said micro-processor being arranged to store the last co-ordinates upon an event of a broken contact with the satellite navigation system.

6. A system according to claim 1, characterized in that the abnormal physiological condition is a cardiac arrest.

7. A portable position indication marker device (PIM), suitable for use in the system as claimed in claim 1, characterized in that said device comprises a position deriving means arranged to derive the position information, a storage unit to store the position information, an identification unit arranged to respond to a linked-up positioning system and a transmitter to transmit the position information to the linked-up positioning system.

8. A portable position indication marker device, as claimed in claim 7, characterized in that the position deriving means comprise an interface arranged to download the position information from a satellite navigation system.

9. A portable position indication marker device, as claimed in claim 7, characterized in that the position deriving means are arranged to download the position information from a terrestrial positioning system.

10. A portable position indication marker device, as claimed in claim 7, characterized in that the position deriving means are arranged to download the position information from a pre-set table of dwell positions stored in the storage unit.

11. A portable position indication marker device, as claimed in claim 7, characterized in that the position deriving means are arranged to read-out a pre-stored position information from a chip-card.

12. A position indication marker according to claim 7, characterized in that said device is programmable by means of a user interface.
